(19) United States
(12) Patent Application Publication
(10) Pub. No.: US 2023/0256158 A1
(43) Pub. Date: Aug. 17, 2023

(54) INFUSION PUMP SYSTEM

(71) Applicant: BD Switzerland Sàrl, Eysins (CH)

(72) Inventors: Lee William TRAVIS, Centennial, CO (US); Brian William WARD, Littleton, CO (US); Jon ANDERSON, Arvada, CO (US)

(21) Appl. No.: 18/015,269

(22) PCT Filed: Jun. 9, 2021
(86) PCT No.: PCT/US2021/036546

§ 371 Date: Jan. 9, 2023

Related U.S. Application Data
(60) Provisional application No. 63/055,792, filed on Jul. 23, 2020.

(51) Int. Cl. A61M 5/14

(52) U.S. Cl. CPC A61M 5/1413; A61M 2205/12; A61M 2205/33

(57) ABSTRACT

Ambulatory modular infusion pump systems are provided, including a housing having a housing front and a housing back, a controller coupled to the housing front, and one or more pump modules disposed within the housing. Each pump module includes a door assembly including a door having a door securement, a channel housing, a cartridge gasket, at least one sensor, a plurality of pump fingers, a frame assembly and a motor. One or more button assemblies are disposed on the housing front, each button assembly having a button disposed adjacent a corresponding pump module and configured to release the door of the corresponding pump module when the button is moved to a release position. Methods of single handed operation of and configuration of ambulatory modular infusion pump systems are also provided.

### TECHNICAL FIELD

The present disclosure generally relates to infusion pumps, in particular ambulatory modular infusion pumps.

### BACKGROUND

Medical fluid transfusion has been widely used in the medical field for intravenous (IV), epidural, and enteral applications. Such fluid transfusion may be provided by a typical infusion pump, such as a large volume pump (LVP). Infusion pumps typically use a linear peristaltic or similar type of mechanism that creates the pumping action by way of squeezing IV tubing to generate the pressure that causes the IV medication to flow through the IV tubing and ultimately into the patient. The squeezing action can also be used as a mechanism to close off the flow. This is used in pumping mechanisms that operate in a cyclical fashion by operating in a cycle that includes a filling portion and a delivery portion. Typical infusion pumps are made for use in relatively stable, clean environments, such as a city hospital or clinic. Further, typical infusion pumps are complex systems that are not designed to be disassembled in the field and require maintenance by highly trained technicians with special tools.

However, infusion pumps are needed in field environments, such as forward military base treatment areas or transport vehicles or aircraft, and require the capability of withstanding vibration, moisture and dust while properly operating. For these reasons, it is desirable to provide a compact ambulatory modular infusion pump system that is hardened against environmental conditions, has easily replaceable pump modules and allows for single handed operation by a medical provider.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient, according to some aspects of the disclosure.

FIG. 2 depicts a perspective view of an ambulatory modular infusion pump system, according to aspects of the disclosure.

FIG. 3 depicts a front view of the ambulatory modular infusion pump system of FIG. 2, according to aspects of the disclosure.

FIG. 4 depicts a rear view of the ambulatory modular infusion pump system of FIG. 2, according to aspects of the disclosure.

FIG. 5 depicts a top view of the ambulatory modular infusion pump system of FIG. 2, according to aspects of the disclosure.

FIG. 6 depicts a bottom view of the ambulatory modular infusion pump system of FIG. 2, according to aspects of the disclosure.

FIG. 7 depicts a side view of the ambulatory modular infusion pump system of FIG. 2, according to aspects of the disclosure.

FIG. 8 depicts an exploded perspective view of an ambulatory modular infusion pump housing, according to aspects of the disclosure.

FIG. 9 depicts an exploded perspective view of an infusion pump module, according to aspects of the disclosure.

FIG. 10 depicts a perspective view of the infusion pump module of FIG. 9, according to aspects of the disclosure.

FIG. 11 depicts another perspective view of the infusion pump module of FIG. 9, according to aspects of the disclosure.

FIG. 12 depicts an exploded perspective partial view of the ambulatory modular infusion pump system of FIG. 2, according to aspects of the disclosure.

FIG. 13 depicts a perspective partial view of the ambulatory modular infusion pump system of FIG. 2 with a tube set cartridge installed, according to aspects of the disclosure.

FIG. 14 depicts a front view of the ambulatory modular infusion pump system of FIG. 2 showing a door of a module in the secured position, according to aspects of the disclosure.

FIG. 15 depicts an exploded perspective view of a door securing mechanism that may be included in a module of the ambulatory modular infusion pump system of FIG. 2, according to aspects of the disclosure.

FIG. 16 is a perspective view of a door securing mechanism that may be included in a module of the ambulatory modular infusion pump system of FIG. 2, according to aspects of the disclosure.

FIG. 17 is a graphical representation of upstream sensor data from operation of the ambulatory modular infusion pump system of FIG. 2, according to aspects of the disclosure.

FIG. 18 is a graphical representation of additional upstream sensor data from operation of the ambulatory modular infusion pump system of FIG. 2, according to aspects of the disclosure.

FIG. 19 is a graphical representation of downstream sensor data from operation of the ambulatory modular infusion pump system of FIG. 2, according to aspects of the disclosure.

FIG. 20 is a graphical representation of additional downstream sensor data from operation of the ambulatory modular infusion pump system of FIG. 2, according to aspects of the disclosure.

FIG. 21 is a graphical representation of upstream/downstream sensor independence data from operation of the ambulatory modular infusion pump system of FIG. 2, according to aspects of the disclosure.

FIG. 22 is a graphical representation of flow data from operation of the ambulatory modular infusion pump system of FIG. 2, according to aspects of the disclosure.

FIG. 23 is a graphical representation of additional flow data from operation of the ambulatory modular infusion pump system of FIG. 2, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

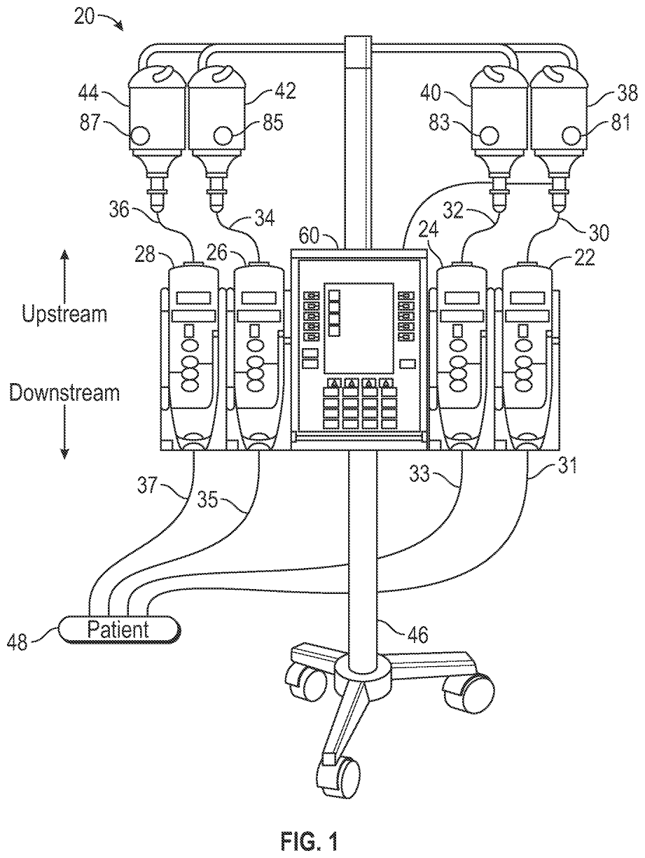

Referring now in more detail to the drawings in which like reference numerals refer to like or corresponding elements among the several views, there is shown in FIG. 1 a patient care system 20 having a controller 60 (e.g., control interface) and four infusion pumps 22, 24, 26, and 28, each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typically, medical fluid administration sets have more parts than are shown in FIG. 1. Many have check valves, drip chambers, valves with injection ports, connectors, and other devices well known to those skilled in the art. These other devices have not been included in the drawings so as to preserve clarity of illustration.

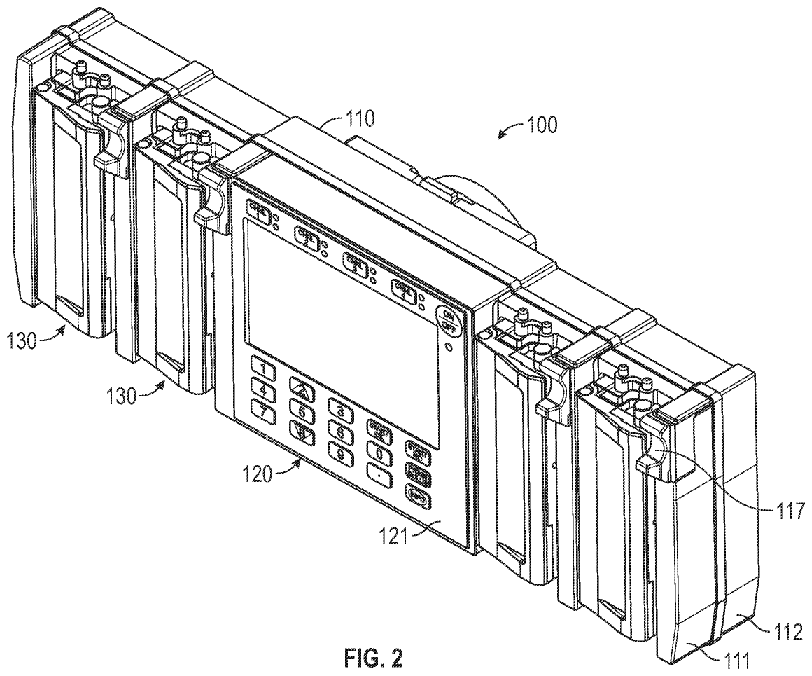

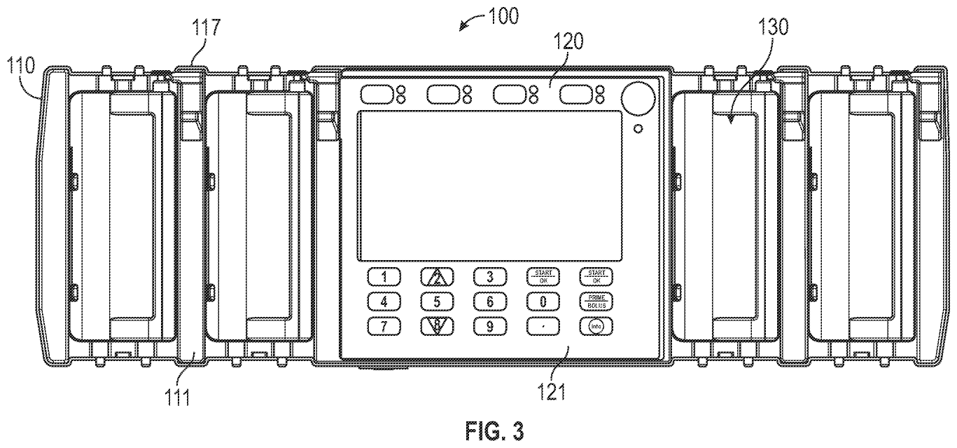

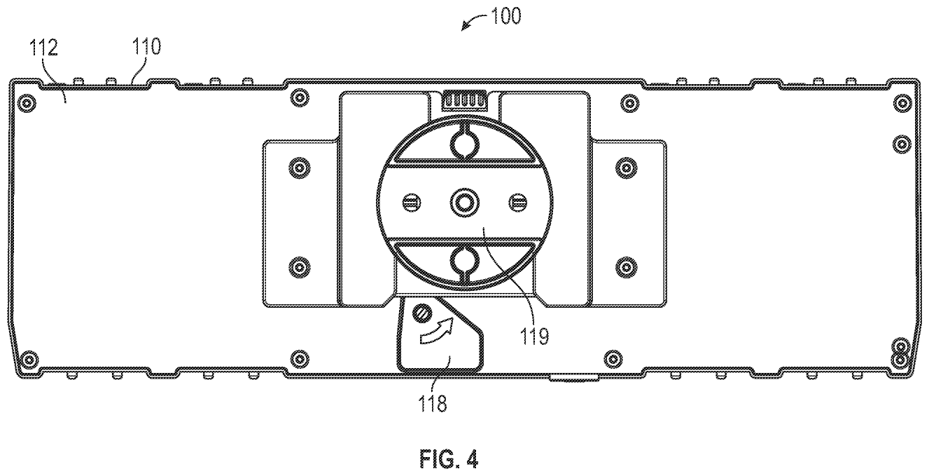

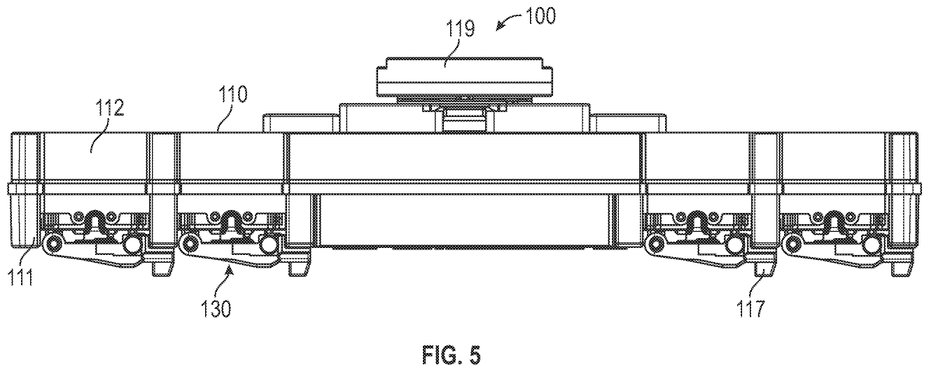

Turning now to FIGS. 2-7, an ambulatory modular infusion pump system 100 is shown in various views. The ambulatory modular infusion pump system 100 includes a housing 110, a controller 120 and pump modules 130. The ambulatory modular infusion pump system 100 is a compact, portable system and may include hardening features, such as shock resistance and/or sealing against moisture and dust. Accordingly, the ambulatory modular infusion pump system 100 is not limited to use in a stable, clean environment (e.g., building based clinic or hospital) and may be used in more rigorous environments in the field (e.g., forward base military treatment center). The ambulatory modular infusion pump system 100 is field serviceable without the need for any special tools and may be operated single handedly by a user (e.g., health care provider). The ambulatory modular infusion pump system 100 may be used for multiple pump configurations. For example, the ambulatory modular infusion pump system 100 may be configured as a two pump system having two pump modules 130 and inserts to block off the spaces in the housing 110 for the other two pump modules 130 to maintain sealing integrity of the system.

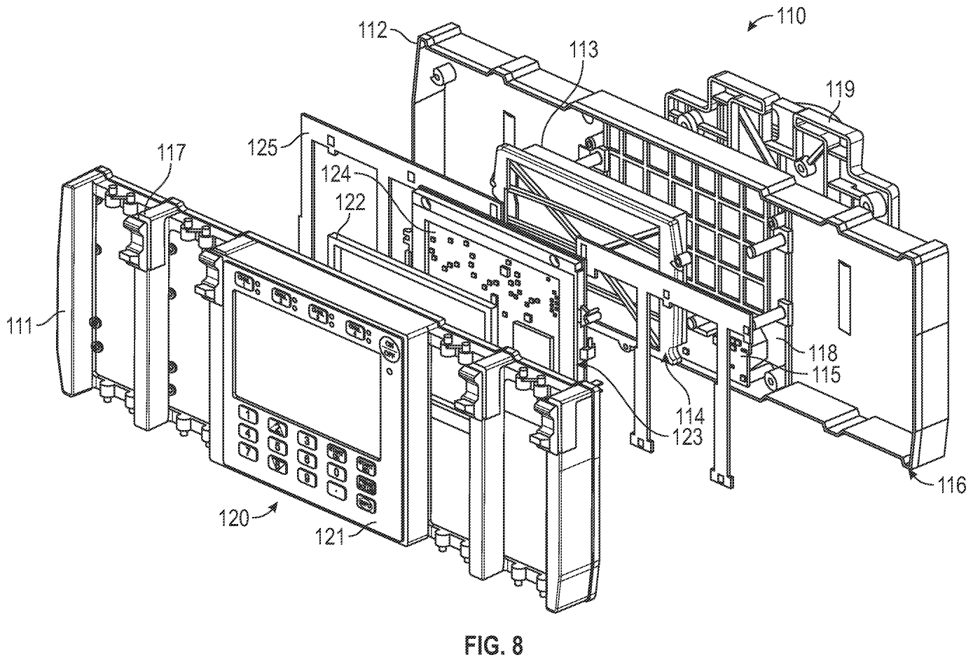

As further shown in FIG. 8, the housing 110 includes a housing front 111 and a housing back 112 on and between which other system components are disposed. For example, the controller 120 may include a keypad 121 that is disposed on the housing front 111. The keypad 121 may be hardened and/or sealed against moisture and dust, and may be usable with chemical resistant gloves. The controller 120 may also include a display 122, a display/board bracket 123 and a processor circuit board assembly 124, each of which is disposed inside the housing 110 between the housing front 111 and the housing back 112.

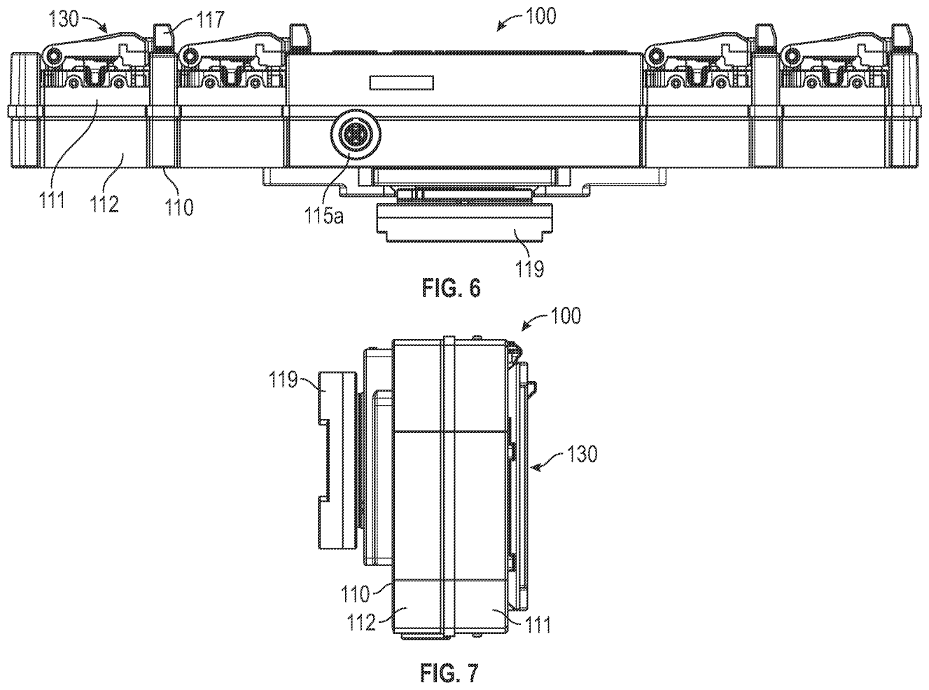

The housing 110 may also include a battery 113 and a battery strap 114 disposed between the housing front 111 and the housing back 112, the battery 113 allowing the ambulatory modular infusion pump system 100 to operate without needing to be plugged in to an external power source. Also disposed between the housing front 111 and the housing back 112 may be cabling 125 for electrically connecting the pump modules 130 and the controller 120. For example, the cabling 125 may be two flat flex cables, each disposed so as to couple with two installed pump modules 130. The inside of the housing 110 may also include a power entry/audio circuit board assembly 115, which provides for charging the battery 113 and for giving audible signals (e.g., alerts, alarms). The power entry/audio circuit board assembly 115 may be coupled to a power connector 115a (see FIG. 6) disposed on a bottom surface of the housing 110. Also included in the inside of the housing 110 is a gasket seal assembly 116 to provide sealing between the housing front 111 and the housing back 112.

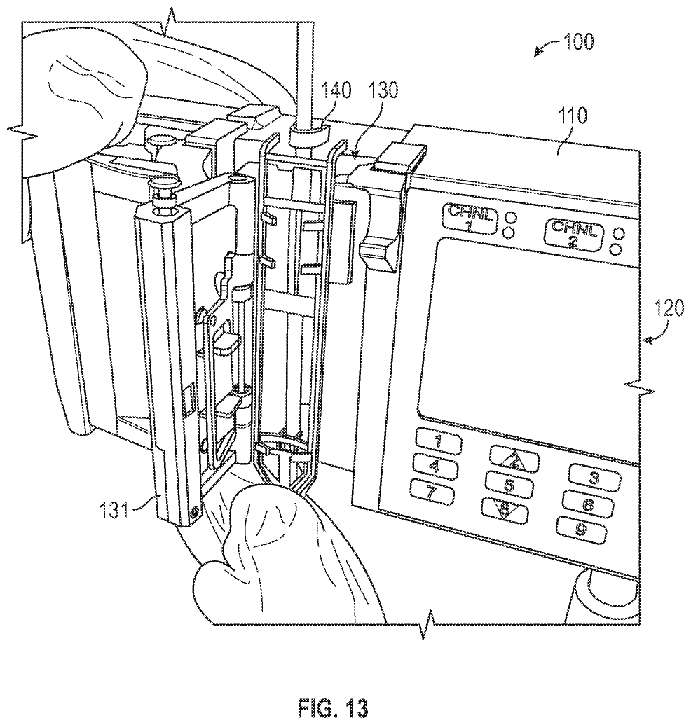

Other system components may be disposed on the outside of the housing 110. For example, button assemblies 117 may be disposed on the housing front 111, one for each pump module 130. The button assembly 117 provides for simple, one handed use by a user to operate the corresponding pump module 130 to loading and unload a tube set 140 (see FIG. 13), such as the tube set cartridge as described in U.S. patent application Ser. No. 16/175,799. For example, the user can lift the button assembly 117 to release the door of the pump module 130, remove the tube set 140 from the pump module 130, set down or dispose of the removed tube set 140, grab a replacement tube set 140, insert the replacement tube set 140 into the pump module 130 and close the door of the pump module 130, all using just one hand. Thus, the user can remove and replace a tube set 140 while maintaining a free hand to achieve another urgent task (e.g., hold pressure on a wound, place and/or hold a ventilation mask).

A connector cover 118 (e.g., USB cover) disposed on the housing back 112 furthers the simplicity of operating the ambulatory modular infusion pump system 100. For example, the user can rotate the connector cover 118 with a thumb or finger in order to plug in an external cable, or vice versa, the user can unplug an external cable and easily rotate the connector cover 118 back to a covering position that protects the sealing integrity of the ambulatory modular infusion pump system 100. A mounting assembly 119 may also be disposed on the housing back 112 to provide for mounting or releasing the ambulatory modular infusion pump system 100 to or from a pole or rail.

Each pump module 130 may be fluidly connected with a fluid source (e.g., 38, 40, 42, 44) via an upstream fluid line (e.g., 30, 32, 34, 36) and may be fluidly connected to a patient via a downstream fluid line (e.g., 31, 33, 35, 37). The pump module 130 may be pre-assembled and calibrated prior to installation into the housing 110. Thus, the ambulatory modular infusion pump system 100 may be configured with one or more pump modules 130 that are already calibrated and ready to use without requiring further calibration after installation into the ambulatory modular infusion pump system 100.

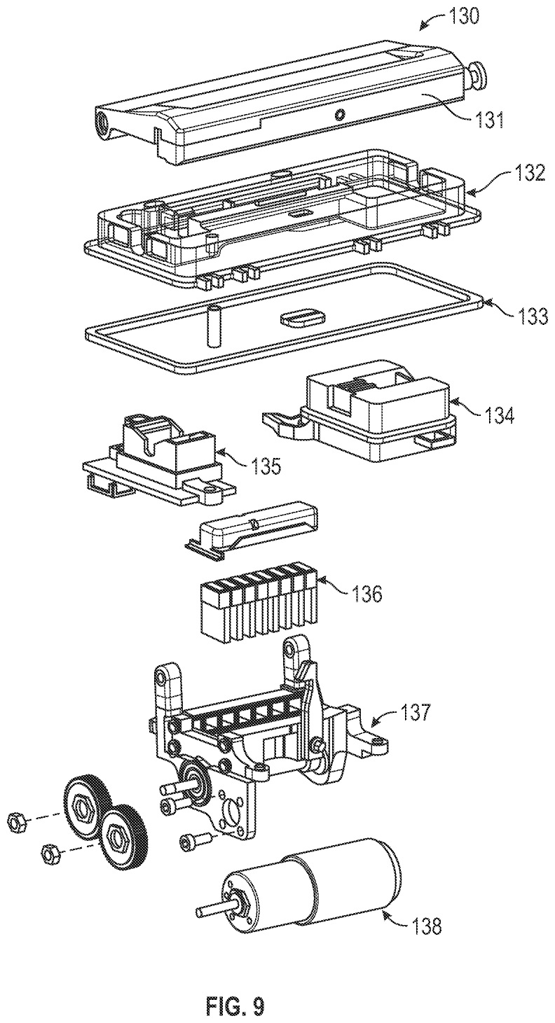

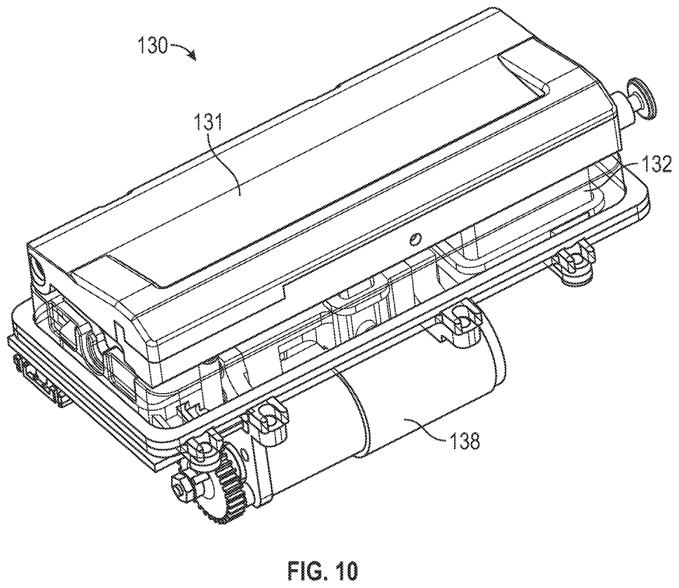

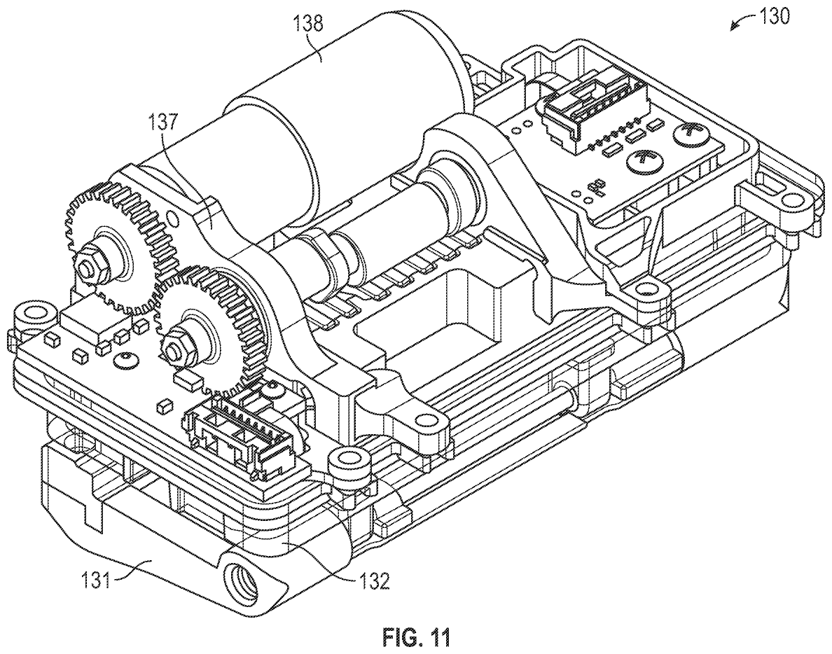

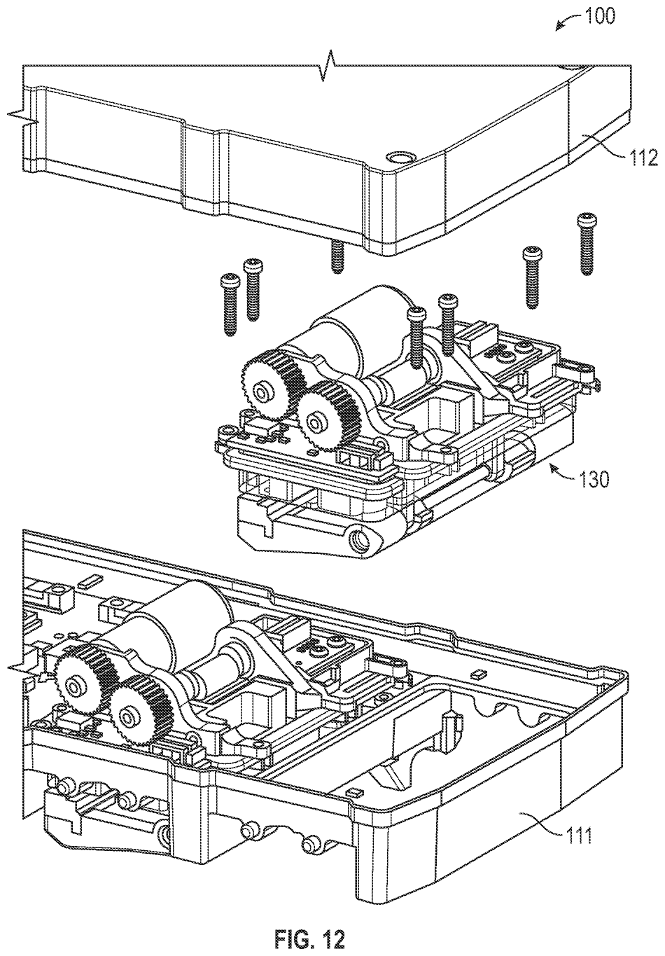

As further shown in FIGS. 9-11, the pump module 130 may include a door assembly 131, a channel housing 132, a cartridge gasket 133, an upstream sensor 134, a downstream sensor 135, pump fingers 136, a frame assembly 137 and a motor 138. The pump module 130 is a complete pumping assembly that can be inserted or dropped into a space in the housing front 111 and the housing back 112 may be coupled to the housing front 111 with simple screws (see FIG. 12). Thus, the pump module 130 may be removed and/or installed without the need for special tools, and once installed the pump module 130 is then ready to receive a tube set 140.

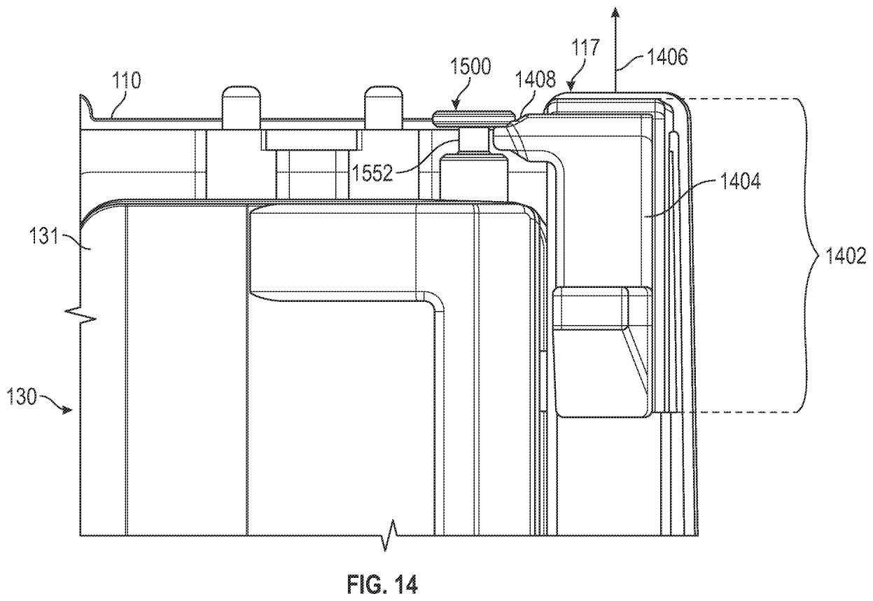

FIG. 14 shows a door assembly 131 of a pump module 130 in the secured position. The button assembly 117 includes an engagement latch 1402 that is coupled to a biased shaft (not shown) which is enclosed within the button assembly 117. The engagement latch 1402 may include a curvature 1404 or other structure (e.g., one or more protrusions to improve friction, loops, etc.) formed to receive a finger and force therefrom. When a force greater than the bias is applied to the biased shaft, the button assembly 117, can be raised to a position above the height of the housing 110. This direction of movement is shown by an arrow 1406 in FIG. 14. If the force is removed, the button assembly 117 may snap back into the position shown in FIG. 14.

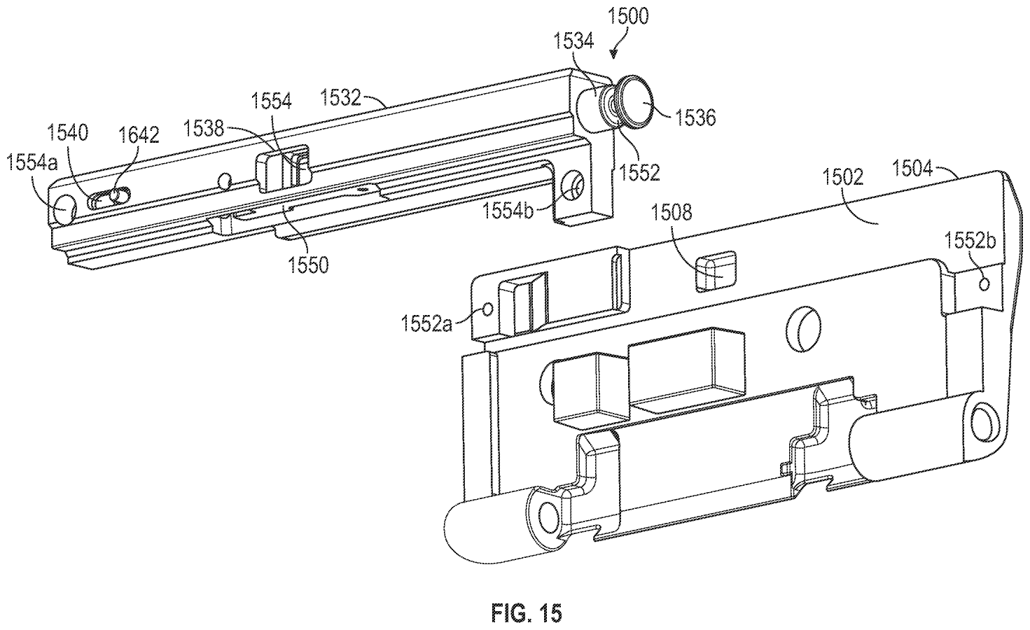

The button assembly 117 includes an engagement portion 1408 that is received by a channel 1552 formed on a door securement 1500. The door securement 1500 includes a shaft with a bias (see, e.g., FIG. 15) that exerts a downward force in the opposite direction of the movement shown by the arrow 1406. This provides an additional point of contact to secure the door assembly 131 of the pump module 130. However, as the button assembly 117 is raised, some force is also transferred to the door securement 1500 via the engagement portion 1408 to also raise at least part of the door securement 1500. Raising the door securement 1500 disengages a latch or other fixture from the door assembly 131 allowing the door assembly 131 of the pump module 130 to open, thereby providing access to the interior of the pump module 130.

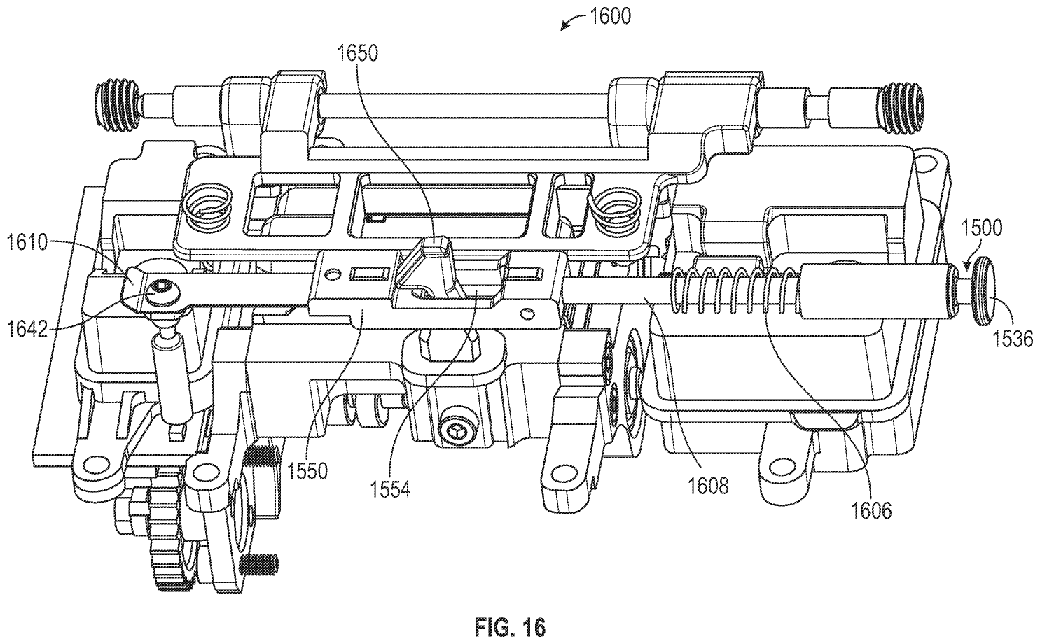

FIG. 15 shows aspects included in a door assembly 131 that may be included in a pump module 130 of the ambulatory modular infusion pump system 100. The door assembly 131 shown in FIG. 15 illustrates the door securement 1500 and an inner face 1502 for a door 1504 of the pump module 130. The inner face 1502 includes securement mount points 1552a and 1552b. Some implementations may include fewer or more securement mount points. The securement mount points 1552a, 1552b may be formed from or include rivets, screws, interlocks, weld points, or other structures to affix the door securement 1500 to the inner face 1502 of the door 1504. Corresponding door mount points 1554a and 1554b are formed on the door securement 1500. Also shown in the door 1504 is a latch deformation 1508. The latch deformation 1508 may receive a portion of a latch that extends from the pump module 130 through the door securement 1500. An example of this extension is illustrated in FIG. 16.

The door securement 1500 includes a housing 1532. The housing 1532 includes a knob channel 1534 through which a knob 1536 extends. The knob 1536 includes the channel 1552 which can engage with the button assembly 117 such as shown in FIG. 14. The housing 1532 includes a latch portal 1538. The latch portal 1538 may receive a portion of a latch 1650 that extends from the pump module 130 (see FIG. 16). The latch 1650 may engage with a slideable carriage 1550 within the housing 1532. The carriage 1550 may include an eye 1554 to which the latch 1650 may engage when the door 1504 is secured. The housing 1532 shown in FIG. 15 also includes a guide groove 1540. A guide pin 1642 may extent into the guide groove 1540 and limit the range of motion for the door securement 1500.

FIG. 16 shows a door securing mechanism 1600 that may be included in a pump module 130 of the ambulatory modular infusion pump system 100. The door securing mechanism 1600 shown in FIG. 16 provides an example of the door securement 1500 and the latch 1650 included in the pump module 130. The latch 1650 is affixed to the pump module 130 and when the door 1504 is in a secured position, the latch 1650 engages with the eye 1554 in the carriage 1550. Also visible in FIG. 16 is a spring 1606 which provides the biasing for the door securement 1500. A shaft 1608 may connect the knob 1536 to the slideable carriage 1602. A flange 1610 may be affixed to a face of the carriage 1550 opposing the face coupled with the shaft 1608. The flange 1610 may include the guide pin 1642. For ease of description, the housing of the door securement 1500 is not shown in FIG. 16.

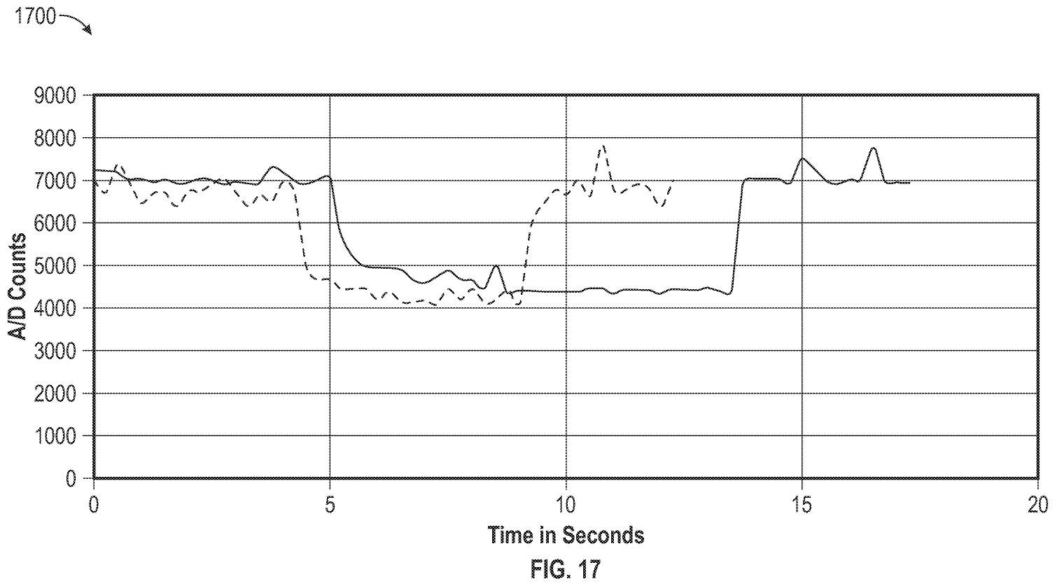

FIG. 17 shows a graphical representation 1700 of test data for the upstream sensor 134 of a pump module 130 during operation of the ambulatory modular infusion pump system 100. During the operation of the ambulatory modular infusion pump system 100, the pump module 130 evacuates fluid from tubing (e.g., tube set 140) disposed within the pump module 130, causing the tubing to compress. The upstream sensor 134 detects distinct differences in readings between when there is no tubing disposed adjacent the upstream sensor 134 and fluid filled tubing is disposed adjacent the upstream sensor 134. The upstream sensor 134 detects occlusion as the tubing decouples from a wall of the upstream sensor 134. The channel housing 132 does not constrain the tubing at the upstream sensor 134, thereby maximizing sensitivity of the upstream sensor 134. The graphical results depict raw pressure profiles versus time for two channels using the same tube set 140.

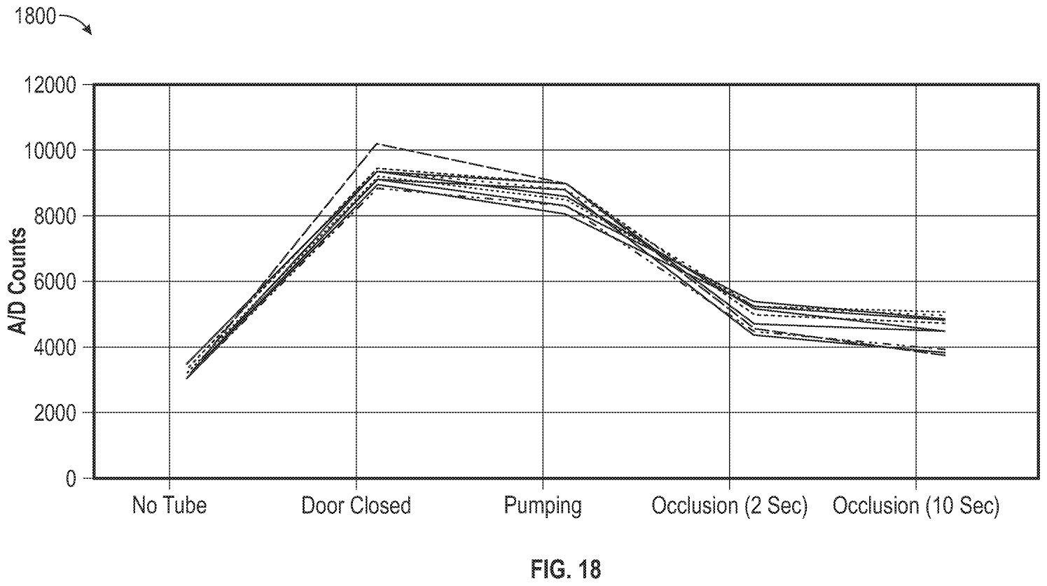

FIG. 18 shows a graphical representation 1800 of additional test data for the upstream sensor 134 of multiple pump modules 130 and tube sets 140 during operation of the ambulatory modular infusion pump system 100, from having no tube disposed in the pump module 130, to closing the door assembly 131 after insertion of a tube set 140, to conducting pumping operations, and to having two occlusion events. ADC values by event are fairly consistent between the channel housings 132 and corresponding tube sets 140. An air sensor may provide for redundant checks. The test data of FIG. 18 shows that each event is easily detectable across channel housings 132 and corresponding tube sets 140. This provides for using a simple calibration method to calibrate each pump module 130.

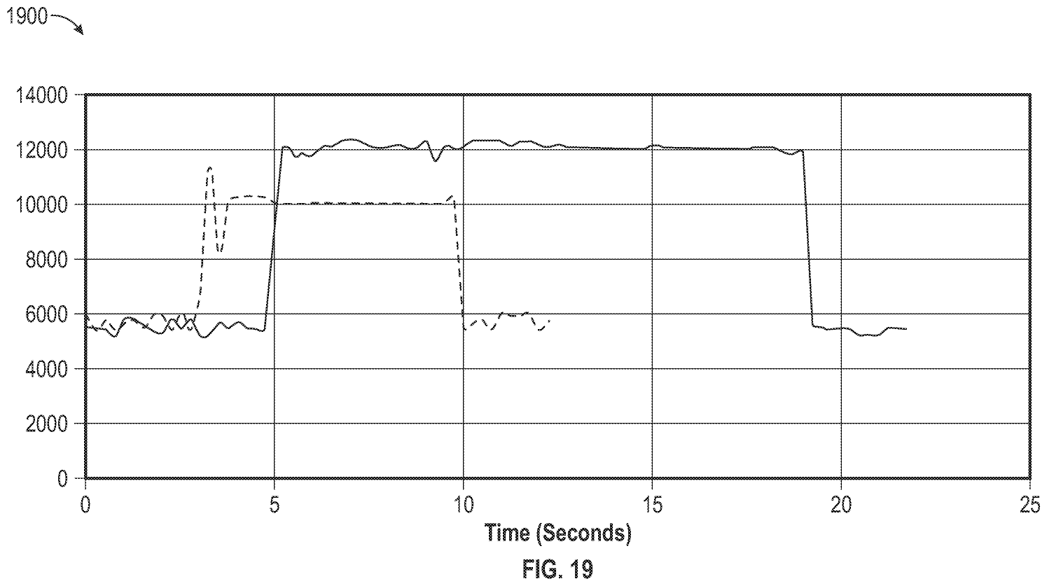

FIG. 19 shows a graphical representation 1900 of test data for the downstream sensor 135 of a pump module 130 during operation of the ambulatory modular infusion pump system 100. During the operation of the ambulatory modular infusion pump system 100, the pump module 130 will quickly build pressure in the tube set 140 in the event of a downstream occlusion. The channel housing 132 constrains the tubing at the downstream sensor 135 to force pressure changes onto the downstream sensor 135, thereby maximizing sensitivity of the downstream sensor 135 to pressure changes. The graphical results depict raw pressure profiles versus time for two channels using the same tube set 140.

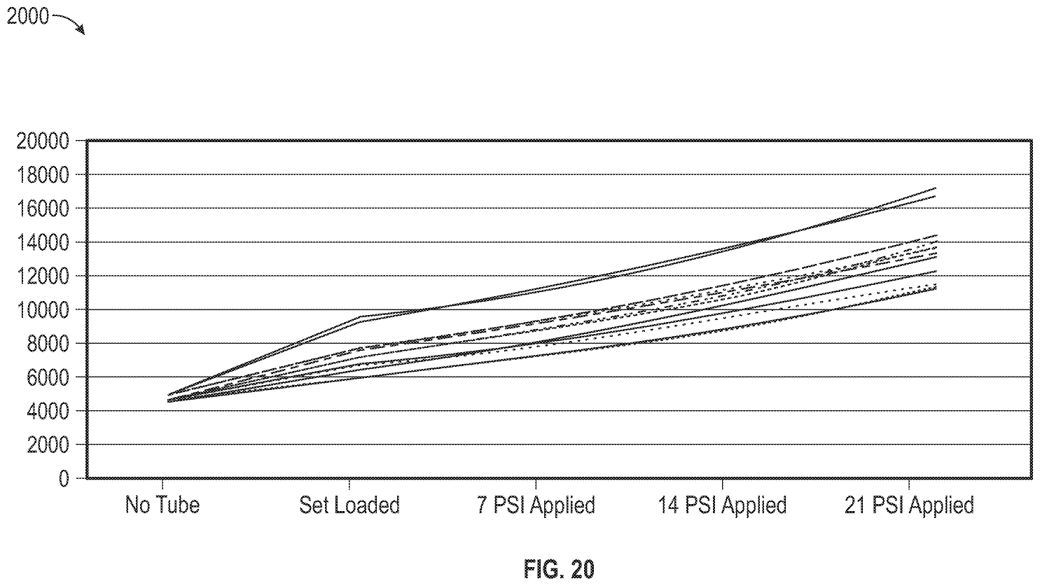

FIG. 20 shows a graphical representation 2000 of additional test data for the downstream sensor 135 of multiple pump modules 130 and tube sets 140 during operation of the ambulatory modular infusion pump system 100, from having no tube disposed in the pump module 130, to loading a tube set 140 and to applying three different pressures. The responses are fairly linear with most non-linearity likely being due to test errors, thus showing that consistent pressure provides for accurate calibration of the pump module 130. Therefore, a percentage change may be a consistent way to determine an occlusion event, particularly at low pumping rates.

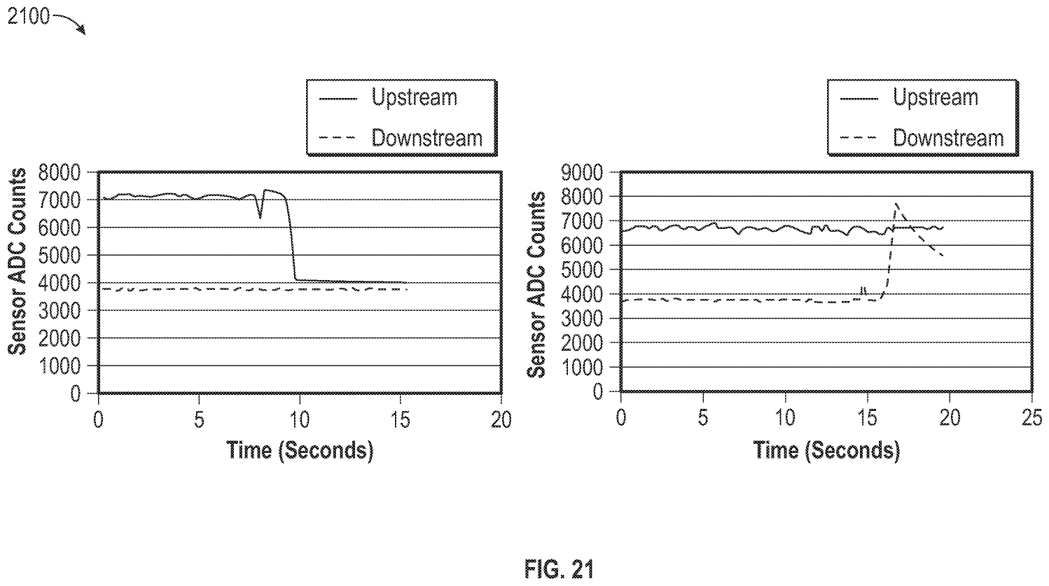

FIG. 21 shows a graphical representation 2100 of test data for sensor independence between the upstream sensor 134 and the downstream sensor 135 of a pump module 130 during operation of the ambulatory modular infusion pump system 100. The test data verifies that upstream and downstream events are independent and checks the ambulatory modular infusion pump system 100 response and pressure variation before/during/after each event. The upstream sensor 134 may vary slightly while the upstream pressure is steady. Pressure is maintained in the downstream tube line even after the pump action has stopped. Thus, release may still be detected to allow for an automatic resume function or to verify the operator has addressed the issue.

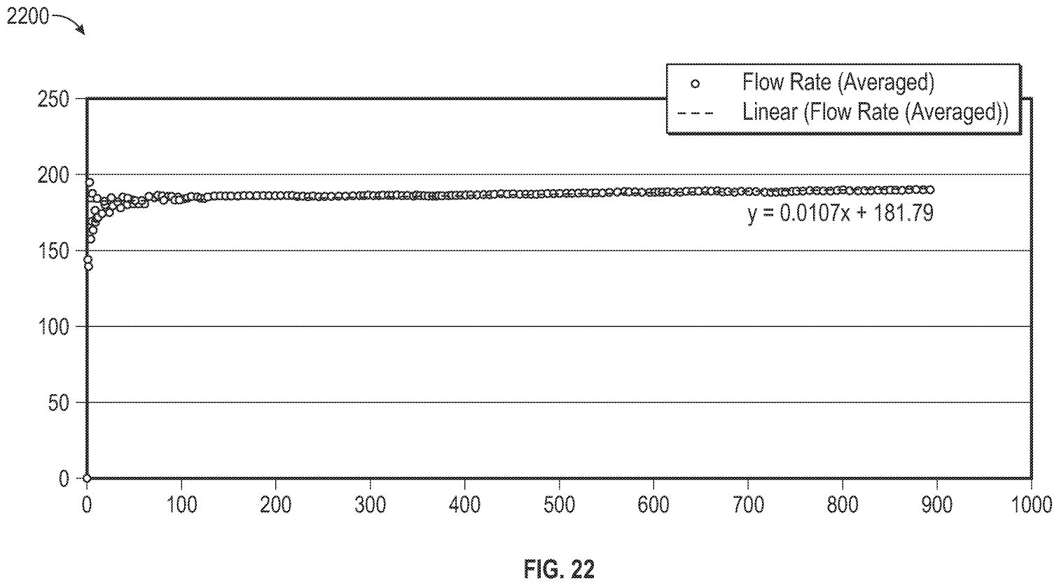

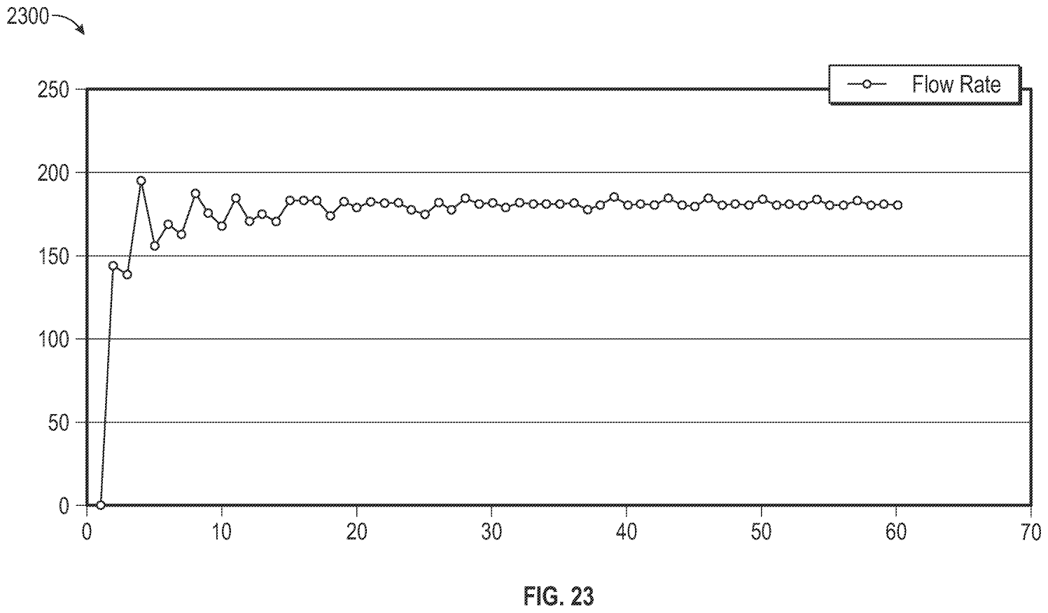

FIGS. 22 and 23 show graphical representations 2200 and 2300 of test data for flow and accuracy of a pump module 130 during operation of the ambulatory modular infusion pump system 100. The results show consistent and linear flow rates throughout operation of the ambulatory modular infusion pump system 100.

Some embodiments of the disclosure are directed to an ambulatory modular infusion pump system. The ambulatory modular infusion pump system includes a housing including a housing front and a housing back, a controller coupled to the housing front, and one or more pump modules disposed within the housing. Each pump module includes a door assembly comprising a door having a door securement, a channel housing, a cartridge gasket, at least one sensor, a plurality of pump fingers, a frame assembly and a motor. The ambulatory modular infusion pump system also includes one or more button assemblies disposed on the housing front, each button assembly having a button disposed adjacent a corresponding pump module and configured to release the door of the corresponding pump module when the button is moved to a release position.

In some aspects of the disclosure, the button assembly includes an engagement latch coupled to a button assembly shaft, wherein the button assembly shaft exerts a downward biasing force on the engagement latch. In some aspects of the disclosure, the engagement latch has a curved surface configured to receive a finger. In some aspects of the disclosure, the button assembly includes an engagement portion configured to be received by a channel disposed on the door securement. In some aspects of the disclosure, the door securement includes a door shaft that exerts a downward biasing force on the engagement portion.

In some aspects of the disclosure, the door assembly includes an inner face of the door, the inner face comprising one or more securement mount points, and the door securement comprising one or more door mount points corresponding to the one or more securement mount points, wherein the door securement is coupled to the inner face via the one or more door mount points mated with the one or more securement mount points. In some aspects of the disclosure, the door includes a latch deformation configured to receive a portion of a latch extending from the pump module. In some aspects of the disclosure, the door securement includes a latch portal disposed in a door securement housing and aligned with the latch deformation, the latch portal configured to receive a portion of the latch extending from the pump module.

In some aspects of the disclosure, the door securement includes a slidable carriage disposed within the door securement housing adjacent to the latch portal. In some aspects of the disclosure, the slidable carriage includes an eye configured to engage with the latch when the door is in a secured position. In some aspects of the disclosure, the door securement includes a guide groove disposed on the door securement housing, the guide groove configured to receive a portion of a guide pin of the door assembly to limit the range of motion of the door securement. In some aspects of the disclosure, the housing includes a battery configured to power the ambulatory modular infusion pump system. In some aspects of the disclosure, the controller comprises at least one of a hardened keypad and a sealed keypad configured to be operated by a chemical resistant glove.

Some embodiments of the disclosure are directed to a sealed ambulatory modular infusion pump system. The ambulatory modular infusion pump system includes a housing including a housing front and a housing back, the housing front having a plurality of spaces, wherein the housing back is secured to the housing front by one or more securing members, and a controller coupled to the housing front. The ambulatory modular infusion pump system also includes one or more pump modules, each pump module removably disposed within one of the plurality of spaces in the housing front, each pump module including a door assembly having a door and a door securement, and an insert coupled to each of the plurality of spaces that do not contain a pump module, each insert configured to provide sealing integrity for that portion of the housing. The ambulatory modular infusion pump system further includes one or more button assemblies disposed on the housing front, each button assembly including a button disposed adjacent a corresponding pump module and configured to release the door of the corresponding pump module when the button is moved to a release position.

In some aspects of the disclosure, each button assembly includes an engagement latch coupled to a button assembly shaft, the button assembly shaft exerting a downward biasing force on the engagement latch, and an engagement portion configured to be received by a channel disposed on the door securement, wherein the door securement comprises a door shaft that exerts a downward biasing force on the engagement portion. In some aspects of the disclosure, the door includes a latch deformation and the door securement includes a latch portal aligned with the latch deformation, the latch deformation and the latch portal each configured to receive a portion of a latch extending from the pump module. In some aspects of the disclosure, the door securement includes a slidable carriage disposed within a door securement housing adjacent to the latch portal, the slidable carriage comprises an eye configured to engage with the latch when the door is in a secured position, and a guide groove disposed on the door securement housing, the guide groove configured to receive a portion of a guide pin of the door assembly to limit the range of motion of the door securement. In some aspects of the disclosure, a battery is configured to power the ambulatory modular infusion pump system and the controller includes a hardened and sealed keypad configured to be operated by a chemical resistant glove.

Some embodiments of the disclosure are directed to a method of single handed operation of any of the above disclosed ambulatory modular infusion pump systems. The method includes lifting one of the button assemblies, releasing the door of the corresponding pump module, if an existing tube set is disposed in the pump module, removing the existing tube set, grasping a replacement tube set, inserting the replacement tube set into the pump module and closing the door of the pump module.

Some embodiments of the disclosure are directed to a method of configuring any of the above disclosed ambulatory modular infusion pump systems. The method includes removing the one or more securing members securing the housing back to the housing front, removing the housing back from the housing front, if an existing pump module is disposed in a selected space in the front housing and a replacement pump module is desired, lifting out the existing pump module from the selected space and setting in the replacement pump module in the selected space, if an existing pump module is disposed in the selected space in the front housing and no pump module in the selected space is desired, lifting out the existing pump module from the selected space and coupling a sealing insert to the selected space, if a sealing insert is coupled to the selected space and a new pump module in the selected space is desired, removing the insert and setting in the new pump module in the selected space, coupling the housing rear to the housing front and replacing the one or more securing members to secure the housing back to the housing front.

As used herein, the terms “control” or “controlling” encompass a wide variety of actions. For example, “controlling” a device may include transmitting one or more messages to adjust an operational state or functional element of the device. The message may include specific instructions to be executed by a processor of the device to manifest the change. The “controlling” may include storing a value in a location of a storage device for subsequent retrieval by the device to be controlled, transmitting a value directly to the device to be controlled via at least one wired or wireless communication medium, transmitting or storing a reference to a value, and the like. For example, a control message may include a value to adjust a level of power from a power source of the controlled device. As another example, a control message may activate or deactivate a structural element of the controlled device such as a light, audio playback, a motor, a lock, a pump, a display, or other component of a device described herein. “Controlling” may include indirect control of the device by adjusting a configuration value used by the controlled device. For example, the control message may include a threshold value for a device characteristic (e.g., temperature, rate, frequency, etc.). The threshold value may be stored in a memory location and referred to by the controlled device during operation.

According to some aspects of the disclosure, a pump assembly includes a fluid flow pump, a tubing pathway configured to receive a fluid tube and a tubing dimension measurement assembly. The tubing dimension measurement assembly includes a processor, an emitter spaced from the tubing pathway and configured to generate an emission into the tubing pathway, and a collector spaced from the tubing pathway, the collector disposed to receive the emission from the emitter, wherein the tubing dimension measurement assembly is further configured to measure an outside diameter (OD) of a tube received in the pathway, wherein said measurement is based at least in part on the emission.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

In any embodiment, data can be forwarded to a “remote” device or location,” where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

Some embodiments include implementation on a single computer, or across a network of computers, or across networks of networks of computers, for example, across a network cloud, across a local area network, on hand-held computer devices, etc. The computers may be physical machines or virtual machines hosted by other computers. In certain embodiments, one or more of the steps described herein are implemented on a computer program(s). Such computer programs execute one or more of the steps described herein. In some embodiments, implementations of the subject method include various data structures, categories, and modifiers described herein, encoded on computer-readable medium(s) and transmissible over communications network(s).

Software, web, internet, cloud, or other storage and computer network implementations of the present invention could be accomplished with standardized programming techniques specifically adapted to cause one or more device to perform the various assigning, calculating, identifying, scoring, accessing, generating or discarding steps described.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An ambulatory modular infusion pump system, comprising:
a housing comprising a housing front and a housing back;
a controller coupled to the housing front;
one or more pump modules disposed within the housing, each pump module comprising:
a door assembly comprising a door having a door securement;
a channel housing;
a cartridge gasket;
at least one sensor;
a plurality of pump fingers;
a frame assembly; and
a motor; and
one or more button assemblies disposed on the housing front, each button assembly comprising a button disposed adjacent a corresponding pump module and configured to release the door of the corresponding pump module when the button is moved to a release position.

2. The ambulatory modular infusion pump system of claim 1, wherein the button assembly comprises an engagement latch coupled to a button assembly shaft, wherein the button assembly shaft exerts a downward biasing force on the engagement latch.

3. The ambulatory modular infusion pump system of claim 2, wherein the engagement latch comprises a curved surface configured to receive a finger.

4. The ambulatory modular infusion pump system of claim 1, wherein the button assembly comprises an engagement portion configured to be received by a channel disposed on the door securement.

5. The ambulatory modular infusion pump system of claim 4, wherein the door securement comprises a door shaft that exerts a downward biasing force on the engagement portion.

6. The ambulatory modular infusion pump system of claim 1, wherein the door assembly comprises:
an inner face of the door, the inner face comprising one or more securement mount points; and
the door securement comprising one or more door mount points corresponding to the one or more securement mount points,
wherein the door securement is coupled to the inner face via the one or more door mount points mated with the one or more securement mount points.

7. The ambulatory modular infusion pump system of claim 6, wherein the door comprises a latch deformation configured to receive a portion of a latch extending from the pump module.

8. The ambulatory modular infusion pump system of claim 7, wherein the door securement comprises a latch portal disposed in a door securement housing and aligned with the latch deformation, the latch portal configured to receive a portion of the latch extending from the pump module.

9. The ambulatory modular infusion pump system of claim 8, wherein the door securement further comprises a slidable carriage disposed within the door securement housing adjacent to the latch portal.

10. The ambulatory modular infusion pump system of claim 9, wherein the slidable carriage comprises an eye configured to engage with the latch when the door is in a secured position.

11. The ambulatory modular infusion pump system of claim 8, wherein the door securement further comprises a guide groove disposed on the door securement housing, the guide groove configured to receive a portion of a guide pin of the door assembly to limit the range of motion of the door securement.

12. The ambulatory modular infusion pump system of claim 1, wherein the housing comprises a battery configured to power the ambulatory modular infusion pump system.

13. The ambulatory modular infusion pump system of claim 1, wherein the controller comprises at least one of a hardened keypad and a sealed keypad configured to be operated by a chemical resistant glove.

14. A sealed ambulatory modular infusion pump system, comprising:
a housing comprising a housing front and a housing back, the housing front comprising a plurality of spaces, wherein the housing back is secured to the housing front by one or more securing members;
a controller coupled to the housing front;
one or more pump modules, each pump module removably disposed within one of the plurality of spaces in the housing front, each pump module comprising a door assembly comprising a door and a door securement;
an insert coupled to each of the plurality of spaces that do not contain a pump module, each insert configured to provide sealing integrity for that portion of the housing; and
one or more button assemblies disposed on the housing front, each button assembly comprising a button disposed adjacent a corresponding pump module and configured to release the door of the corresponding pump module when the button is moved to a release position.

15. The sealed ambulatory modular infusion pump system of claim 14, wherein each button assembly comprises:
an engagement latch coupled to a button assembly shaft, the button assembly shaft exerting a downward biasing force on the engagement latch; and
an engagement portion configured to be received by a channel disposed on the door securement, wherein the door securement comprises a door shaft that exerts a downward biasing force on the engagement portion.

16. The sealed ambulatory modular infusion pump system of claim 14, wherein the door comprises a latch deformation and the door securement comprises a latch portal aligned with the latch deformation, the latch deformation and the latch portal each configured to receive a portion of a latch extending from the pump module.

17. The sealed ambulatory modular infusion pump system of claim 16, wherein the door securement comprises:
a slidable carriage disposed within a door securement housing adjacent to the latch portal, the slidable carriage comprises an eye configured to engage with the latch when the door is in a secured position; and
a guide groove disposed on the door securement housing, the guide groove configured to receive a portion of a guide pin of the door assembly to limit the range of motion of the door securement.

18. The sealed ambulatory modular infusion pump system of claim 16, further comprising a battery configured to power the ambulatory modular infusion pump system, and the controller comprises a hardened and sealed keypad configured to be operated by a chemical resistant glove.

19. A method of single handed operation of the ambulatory modular infusion pump system of claim 1, the method comprising:
lifting one of the button assemblies;
releasing the door of the corresponding pump module;
if an existing tube set is disposed in the pump module, removing the existing tube set;
grasping a replacement tube set;
inserting the replacement tube set into the pump module; and
closing the door of the pump module.

20. A method of configuring the ambulatory modular infusion pump system of claim 14, the method comprising:
removing the one or more securing members securing the housing back to the housing front;
removing the housing back from the housing front;
if an existing pump module is disposed in a selected space in the front housing and a replacement pump module is desired, lifting out the existing pump module from the selected space and setting in the replacement pump module in the selected space;
if an existing pump module is disposed in the selected space in the front housing and no pump module in the selected space is desired, lifting out the existing pump module from the selected space and coupling a sealing insert to the selected space;
if a sealing insert is coupled to the selected space and a new pump module in the selected space is desired, removing the insert and setting in the new pump module in the selected space;
coupling the housing rear to the housing front; and
replacing the one or more securing members to secure the housing back to the housing front.
